(19) United States
(12) Patent Application Publication
(10) Pub. No.: US 2021/0322058 A1
(43) Pub. Date: Oct. 21, 2021

(54) Insert for Convertibly Anchoring an Artificial Ligament Tether in a Tulip of a Spinal Fixation Construct

(71) Applicant: SII Limited, New Albany, OH (US)

(72) Inventor: Paul H. Eichenseer, New Albany, OH (US)

(21) Appl. No.: 16/849,033

(22) Filed: Apr. 15, 2020

(51) Int. Cl. A61B 17/70

(52) U.S. Cl. CPC A61B 17/7022; A61B 17/701

(57) ABSTRACT

An insert for convertibly anchoring an artificial ligament tether in a tulip of a spinal fixation construct. A prior art tulip has a socket with opposite end openings and configured in size and shape to receive a spinal longitudinal support rod. A tulip also has a clamping screw threadedly engaged to the tulip to clamp a support rod in the socket. The invention is an insert body having an exterior configured to conformingly seat against a surface of the tulip socket and also having a passageway extending entirely through the insert body in a direction to extend between the end openings of the tulip. A surgeon inserts a tether through the passageway and rotates the clamping screw to translate the clamping screw against the insert body. Rotation of the clamping screw clamps the tether to the insert body and clamps the insert body to the tulip.

### BACKGROUND OF THE INVENTION

This invention is directed to a tether-receiving insert that, in a surgical procedure, can be mounted in a tulip that is part of a spinal fixation construct and used for anchoring a tether to the construct but, in a later surgical procedure, can be removed to permit the tulip to be converted to its conventional purpose of connection to a spinal fixation rod.

A common treatment for one or more degenerated, deformed or damaged vertebral stages of a patient's spinal column is internal spinal fixation. Typically, this involves the surgical attachment of a spinal implant system to provide a construct that is attached to two or more adjacent vertebrae to support and stabilize the vertebrae in order to allow them to fuse together in a stationary relationship relative to each other. Spinal fusion constructs typically include pedicle screws and longitudinal support members which are rods that are attached to the pedicle screws. Together the pedicle screws and the rods fix the position of the adjacent vertebrae to which they are attached. Spinal fixation surgery sometimes additionally includes posterior ligament reconstruction using artificial ligament tape, or other cordage, to form a tether in a construct for the purpose of tethering an uppermost fused vertebra to higher vertebrae.

An important component of many spinal fixation constructs is a tulip. A tulip is a body having a socket, typically formed as a saddle or a cylindrical hole, for receiving a longitudinal support rod and also having a cooperating clamping screw for clamping the rod rigidly in the tulip. A diverse variety of monoaxial and polyaxial tulips are commonly constructed on pedicle screws but are also a component of other orthopedic implant devices.

In addition to the use of tulips on pedicle screws, the prior art has also provided revision connectors also described as rod-to-rod connectors. Those connectors include multiple tulips attached together, usually as a unitary body. Some rod-to-rod connectors are called W-connectors or double slot heads because they are two parallel-oriented, side-by-side tulips formed on a unitary body. Rod-to-rod connectors allow the surgeon to attach a first tulip of a connector to a rod and then attach a second tulip of the connector to a different rod.

Sometimes a problem develops in patients after spinal fixation surgery as a result of patient activity or stature. For example, the vertebra that is immediately above the highest fused vertebra may break away from the highest fused vertebra and fall forward. Surgeons may attempt to prevent that or other problems by securing vertebrae that are immediately above the uppermost instrumented vertebra to the uppermost instrumented vertebra by means of an artificial ligament tether. (An instrumented vertebra is a vertebra to which a device is implanted or connected.) The term “tether” is used in a generic sense to refer to a type of cordage that is available for surgeons to perform this procedure. Some prior art refers to equivalent structures as tape, cable, rope, tether, wire, braid, band or strand. They are an elongated structure that is flexible so that they bend easily (with the application of relatively little force) but also have a strong resistance to being stretched longitudinally by a substantial pulling force. Other equivalent terms include artificial ligament reconstruction tape, Mersilene® tape and TLS® strips.

One way of applying a tether is to first drill a hole through the spinous process of the uppermost instrumented vertebra and also through the contiguous vertebrae that are immediately above the uppermost instrumented vertebra and are to be secured by the tether. The tether is then threaded through the drilled holes upward from the uppermost instrumented vertebra to the higher vertebrae and then threaded back down again through the holes to the uppermost instrumented vertebra. In one procedure the tether is then pulled in tension and tied to itself or to one or two of the rods that are a part of the construct. Unfortunately, this procedure often results in a loosening of the ligament tape because much of the tension is lost during the manipulation of the ligament tape into a knot and also because the tape can loosen further after surgery from slippage of the knot.

In order to reduce this loosening problem, the prior art has provided specialized anchoring devices that are attached to the construct and allow the surgeon to fasten the ligament tape to the construct. These specialized anchoring devices avoid the use of a knot and also allow a greater tension to be maintained while the tether is being secured to the anchor and reduce or eliminate later slippage at the anchor. Examples of specialized anchoring devices are found in U.S. Application Publications US 2014/0257397 and US 2018/0078286. Special purpose anchoring devices have a component that connects to a pedicle screw, a rod or other component of the construct and have another component for attachment to the tether.

In some patients who receive spinal fixation surgery that included the use of a tether as described above, a subsequent revision surgery is needed. For example, revision surgery may be needed as a result of subsequent disease progression or a patient's excessive physical activity that causes the vertebra that is immediately above the highest fused vertebra to break away from the highest fused vertebra and fall forward. In that event it becomes desirable to have a surgeon perform a surgical revision that extends the initial spinal fusion to additional vertebrae, including at least any vertebra to which the tether was previously connected.

In performing the revision surgery, the surgeon releases the previously installed tether from the device to which it is anchored and removes the tether from the patient. The construct of the initial spinal fixation can be extended by attaching one or more additional longitudinal support rods to the existing construct of the initial spinal fixation. One way to do that is for the surgeon to connect one or more rod-to-rod connectors of the type described above to the existing, previously installed longitudinal support rods. Then a new support rod can be connected to the rod-to-rod connector. If the initially installed tether was anchored to a specialized anchoring device, such as described above, the surgeon must first remove that anchoring device before installing a rod-to-rod connector.

The object and purpose of the invention is to provide an anchor for a tether that can be used in an initial spinal fixation construct but also reduces the manipulative operations that are needed for a revision surgery that extends the construct.

Another object and purpose of the invention is to provide a structure that permits a surgeon to anchor an artificial ligament tether to a tulip but also permits the structure to be removed in a surgical revision so that the tulip can be converted to use for attachment to a rod for extending the initial spinal fusion to additional vertebrae.

Yet another object and purpose of the invention is to provide an insert that, in a spinal fixation surgery, allows a surgeon to attach one tulip of a rod-to-rod connector to a rod, to attach the insert of the invention in a second tulip of the rod-to-rod connector and finally to attach an artificial ligament tether to the insert, but also, in a later revision surgery, allows the surgeon to remove the insert and use the second tulip for attachment to a rod. This avoids the need during the revision surgery to remove a previously installed special purpose tether connector and also avoids the need to install a rod-to-rod connector.

A further object and purpose of the invention is to provide a tether anchoring insert that can be attached in a tulip in any surgical implant device in the same manner that the tulip is designed to be attached to a rod by using the tulip's clamping screw and can be removed in a subsequent revision surgery so the tulip can be repurposed for supporting a rod.

### SUMMARY OF THE INVENTION

An insert for convertibly anchoring an artificial ligament tether in a tulip of a spinal fixation construct. Prior art tulips have a socket with opposite end openings and are configured in size and shape to receive a spinal longitudinal support rod. Tulips also have a clamping screw threadedly engaged to the tulip to clamp a support rod in the socket. The invention is an insert body having an exterior configured to conformingly seat against a surface of the tulip socket and also having a passageway extending entirely through the insert body in a direction to extend between the end openings of the tulip. A surgeon inserts a tether through the passageway and rotates the clamping screw to translate the clamping screw against the insert body. Rotation of the clamping screw clamps the tether to the insert body and clamps the insert body to the tulip.

### BRIEF DESCRIPTION OF DRAWINGS

FIG. 1 is a front view of a prior art pedicle screw with a longitudinal support rod in the socket of the pedicle screw.

FIG. 2 is a side view of the pedicle screw of FIG. 1.

FIG. 3 is a top view of the pedicle screw of FIG. 1.

FIG. 4 is an exploded view in perspective of a prior art tulip and an associated support rod.

FIG. 5 is a top view of a prior art rod-to-rod connector.

FIG. 6 is a front view of the rod-to-rod connector of FIG. 5.

FIG. 7 an end view of an insert embodying the invention.

FIG. 8 is a top view of the embodiment of FIG. 7.

FIG. 9 is a view in perspective of the embodiment of FIG. 7.

FIG. 10 is an end view of the embodiment of FIG. 7 after a tether has been clamped in its passageway.

FIG. 11 is front view of a rod-to-rod connector, like the connector of FIG. 5, with the embodiment of FIG. 7 inserted in one of its tulips.

FIG. 12 is a front view of the rod-to-rod connector of FIG. 11 after a tether has been clamped in an insert embodying the invention.

FIG. 13 is a side view of an insert embodying the invention that also has a head at one end.

FIG. 14 is a side view of an insert embodying the invention that has two heads, at one each end.

FIG. 15 is an end view of an alternative embodiment of the invention.

FIG. 16 is an end view of another alternative embodiment of the invention.

FIG. 17 is an end view of another alternative embodiment of the invention.

FIG. 18 is an end view of another alternative embodiment of the invention.

FIG. 19 is an end view of another alternative embodiment of the invention.

FIG. 20 is an end view of another alternative embodiment of the invention.

FIG. 21 is an end view of another alternative embodiment of the invention.

FIG. 22 is an end view of another alternative embodiment of the invention.

FIG. 23 is an end view of another alternative embodiment of the invention.

In describing the preferred embodiment of the invention which is illustrated in the drawings, specific terminology will be resorted to for the sake of clarity. However, it is not intended that the invention be limited to the specific term so selected and it is to be understood that each specific term includes all technical equivalents which operate in a similar manner to accomplish a similar purpose.

### DETAILED DESCRIPTION OF THE INVENTION

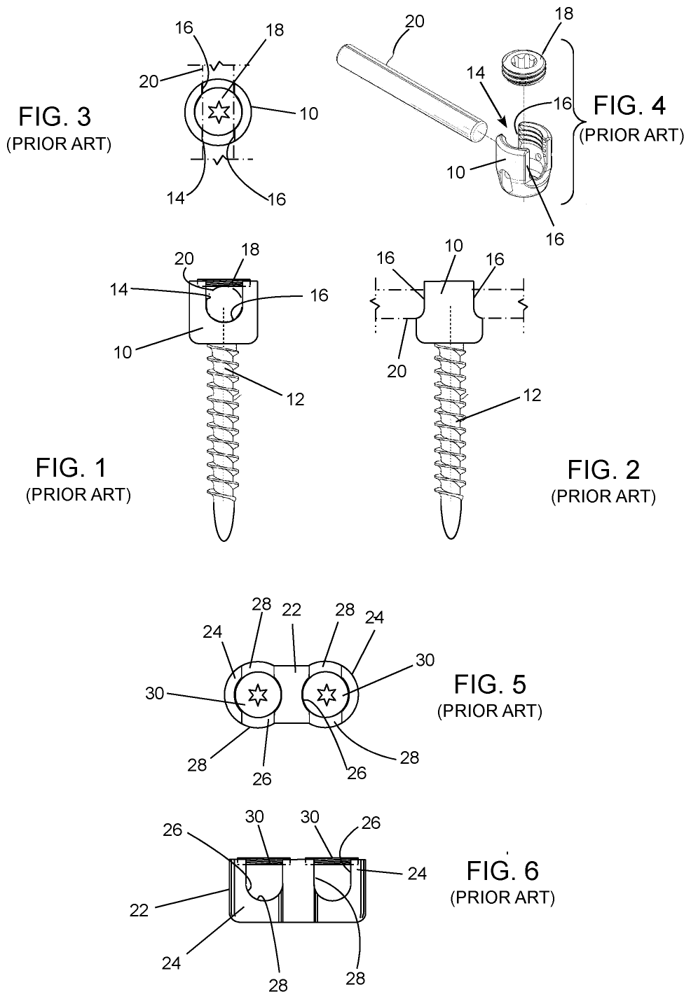

Because the invention is a tether-anchoring insert that is designed and configured in size and exterior shape so it can be clamped in a tulip, FIGS. 1 through 6 illustrate tulips, which are a well-known, prior art component of various prior art orthopedic implant devices. FIGS. 1-3 illustrate a pedicle screw which has a tulip 10 attached to the end of a threaded shank 12. FIG. 4 shows an example of a tulip for a polyaxial pedicle screw. The same reference numbers are used in FIGS. 1-4 for designating the tulips and their component parts. The tulip 10 has a socket 14 with opposite end openings 16 and a clamping screw 18. The purpose of the tulip 10 is to receive a longitudinal support rod 20 in its socket 14. The clamping screw 18 is threadedly engaged in the tulip so it can be rotated to clamp the rod 20 in the socket 14. Support rods are manufactured in a series of standard sizes and shapes, most commonly cylindrical. Therefore, the sockets of tulips are also configured in size and shape to conformingly and matingly receive a standard rod 20.

Commercially available orthopedic implant devices that have a tulip are manufactured with a variety of tulip socket and tulip clamping screw designs and configurations. FIGS. 1-4 illustrate the most common socket which has a lateral slot that forms a saddle. The saddle is contoured to seat against the rod with mating interfacing contours. As shown in FIGS. 1-4, the most common socket has semi-cylindrical surfaces to seat tightly against a cylindrical surface of a rod. Other sockets are a lateral hole, usually circular in cross section, through which the rod is slid by the surgeon. A common type of clamping screw is a set screw 18 as shown in FIGS. 1-4. The set screw 18 has male threads which threadedly engage female threads on the interior surface of the socket 14. Other tulips have a clamping screw that is a nut with female threads that threadedly engage male threads formed on the exterior surface of the tulip. Some tulips, which have a lateral hole instead of a slot, have a threaded screw that is threadedly engaged in a port through the top of the tulip so the screw can be rotated into the lateral hole to clamp a rod against a wall of the hole. In view of the many variations in tulip design, the term “clamping screw” is used as a generic term for the part of the tulip that moves axially when rotated to clamp a support rod against the bottom of the socket. The term includes structures that use other than conventional threads, such as inclined grooves on opposite sides of the saddle and an internal screw with outward extending tabs that extend into the grooves for moving into the saddle when rotated. Such structures have been referred to as a twist lock. The term also includes other tulip designs that use a surrounding nut that is threadedly engaged to the exterior of the tulip assembly and an interior disk with diametrically opposite arms that extend outwardly through open ends of the socket so the arms are contacted by the nut and the disk is driven against the rod when the nut is rotated.

A pedicle screw is not the only orthopedic implant device that includes a tulip. FIGS. 5 and 6 illustrate a type of rod-to-rod connector 22. The rod-to-rod connector 22 has two tulips 24 that are rigidly connected together as part of a unitary body. Each tulip 24 has a socket 26 with opposite end openings 28. Rod-to-rod connectors are available with more than two tulips. They also are available with side-loading and top loading orientations of their tulips and with “closed” rod sockets (circular holes). A rod-to-rod connector provides a way for a surgeon to anchor one or more rods on another rod instead of on a pedicle screw. Consequently, their tulips are configured in size and shape to matingly receive standard spinal longitudinal support rods. As with other tulips, each tulip 24 of a rod-to-rod connector includes a clamping screw 30 that is threadedly engaged to the tulip and configured to clamp a support rod in its socket.

The invention is a tether-receiving insert that can be inserted and clamped in a tulip whether the tulip is a component of a rod-to-rod connector, a pedicle screw or any other surgical implant. The insert of the invention can be clamped in a tulip in the same manner that the tulip is designed to clamp to a rod. The insert allows a tether to be anchored to the insert and therefore to other parts of a construct that are rigidly connected to the tulip. One advantageous and novel utility of the invention is that it allows a surgeon to connect one tulip of a rod-to-rod connector to a first rod in a construct and connect an insert embodying the invention in the second tulip of the rod-to-rod connector. The benefit of that utility is that, if a later surgical revision is ever needed in order to extend the spinal fusion, the tether-receiving insert of the invention can be removed and a second extension rod can be substituted for the insert in the second tulip. That utility reduces the amount of reconstruction of the original construct that is necessary in later surgical revision surgery.

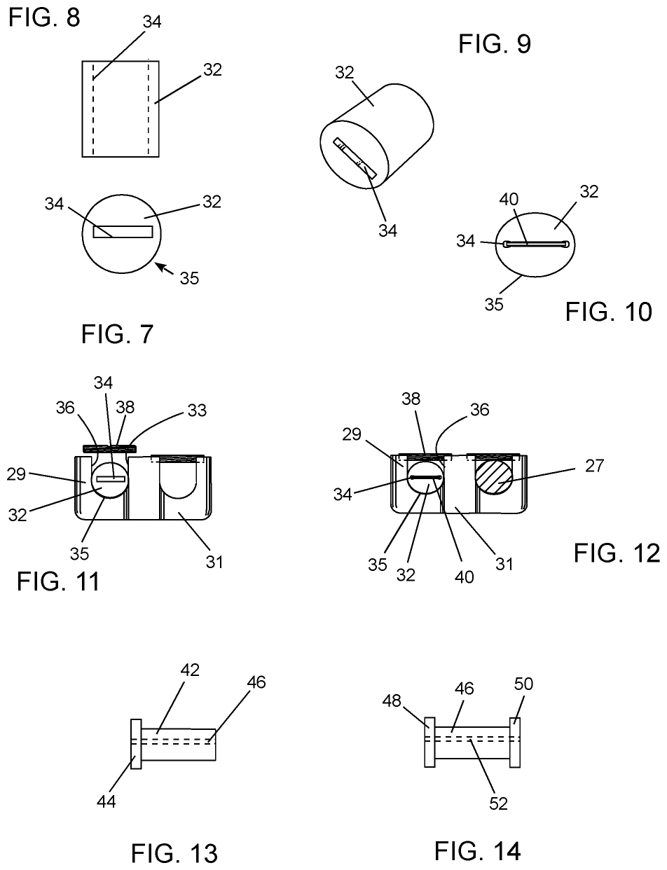

A variety of alternative embodiments of the invention are illustrated in FIGS. 7 through 23. FIGS. 7-10 illustrate an example of an insert of the invention and FIGS. 11 and 12 show the mounting of that insert in a tulip 29 that is a part of a rod-to-rod connector 31. The insert of FIGS. 7-10 is an insert body 32 having an exterior that is configured in size and shape to conformingly seat against a surface of the socket 33 component of the tulip 29. This means that the bottom surface 35 of the insert's exterior, which seats against the bottom of the socket 33 in the insert body 32, should have a contour that allows the insert body 32 to be inserted into the socket 33 and clamped to seat against the bottom surface of the socket 33 in a manner that does not permit lateral or pivotal slippage of the insert body 32 within the socket 33. The most common shape of the interfacing surfaces that seat together is the surface of a cylindrical sector, usually a semi-cylindrical surface.

The insert body 32 has a passageway 34 extending entirely through the body 32 in a direction so that, when mounted in the tulip 29, the passageway 34 extends between the end openings 36 of the tulip 29. With the most common tulips, the passageway is parallel to the axis of the cylindrically contoured surface of the insert body. The passageway 34 is configured to receive a tether. The configuration of the passageway 34 requires that the passageway have a size and cross-sectional shape that permits a surgeon to manually insert a tether into the passageway 34 and manually push the tether through the passageway 34. The illustrated passageway 34 is rectangular in cross-section so that a tether in the shape of a tape or ribbon can be passed through the passageway 34. If the tether is a cord or wire of circular cross-section the passageway can likewise have a circular cross-section. The insert body 32 and the position and orientation of the passageway 34 must be configured so that rotation of the tulip's clamping screw 38 translates the clamping screw 38 against the insert body 32, clamps the insert body in the tulip 29, and clamps a tether, which is inserted through the passageway 34, to the insert body 32.

An insert of the type illustrated in FIGS. 7-12 requires that the insert body be formed of a malleable or compliant material, preferably commercially pure titanium, so that the force exerted by the clamping screw 38 when rotated will deform the insert body to squeeze interior walls of the passageway 34 against the inserted tether. The combination of force and malleability must be sufficient to fix the tether in place so that it will not slide within the passageway 34 from any longitudinal force that is applied to the tether of a magnitude that can be expected to be encountered in normal human activity by the receiving patient.

In the most preferred embodiments of the invention, the tulip and the insert body have conformingly mating, cylindrically contoured surfaces. The contoured surfaces are clampable in the tulip's socket in interfacing contact against each other by the clamping screw. The contoured surfaces are clamped in an orientation with the cylindrically contoured surface of the insert body extending between the end openings of the tulip. A cylindrically contoured surface is the curved surface of a solid cylindrical sector. The most preferable cylindrically contoured surface is the curved surface of a semi-cylinder with the radius of a standard rod so that the insert is universal; that is, so the insert body can be inserted in the tulip sockets from different manufacturers of tulips on surgical implants and be seated in a socket in the manner of a rod. The conformingly mating, cylindrically contoured surfaces that come into interfacing contact are the surfaces of the insert body and of the tulip socket that are distal from the clamping screw when the insert body is clamped in the tulip. In other words, the first one of the conformingly mating, cylindrically contoured surfaces that come into interfacing contact is the bottom interior surface of the saddle or slot of the tulip or the distal part of a tulip that has a circular hole instead of a saddle or slot. The other conformingly mating, cylindrically contoured surface is the surface of the insert body that seats against the first one of the conformingly mating, cylindrically contoured surfaces.

FIGS. 10 through 12 illustrate installing the insert body 32 into the tulip 29 of the rod-to-rod connector 31 and securing a tether to the insert body 32 and to the tulip 29. A rod 27 is also mounted in the connector 31 in the conventional manner. The insert body 32 is first positioned in the tulip 29 as shown in FIG. 11. The clamping screw 38 may then be partially screwed into the tulip 29 but not tightened fully against the insert body 32. A tether 40 is then inserted through the passageway 34 and pulled and held in an appropriate tension. With the tether 40 held in tension, the clamping screw 38 is rotated and tightened against the insert body 32 with sufficient torque to deform the insert body 32 and compress the inner walls of the passageway 34 securely against the tether as illustrated in FIG. 12. FIG. 10 is an enlarged view of the insert body 32 with the tether 40 clamped in the passageway 34 of the deformed insert body 32.

FIG. 13 illustrates a cylindrical insert body 42 that has a concentric circular head 44 at one end. The head 44 is radially enlarged beyond the size of the opposite end openings of the tulip in which it will be installed. The insert body 42 also has a longitudinal passageway 45 that extends entirely through the insert body 42 parallel to the axis of the insert body 42. FIG. 14 illustrates an insert body 46 that has heads 48 and 50, one at each of its opposite ends, and also has a longitudinal passageway 52. Both heads 48 and 50 are enlarged beyond the size of the opposite end openings of the tulip in which the insert body 46 will be installed. The heads 48 and 50 are spaced apart from each other by a distance that is at least as far as the distance between the end openings of the tulip in which the insert body 46 will be installed.

The head 44 and the heads 48 and 50 prevent their insert body from slippage by sliding along or out of its tulip socket in the event that a force is applied to a tether in an axial direction. From that function, it is apparent that it is not necessary that the head 44 or the heads 48 and 50 be circular or even have a symmetrical or regular shape. Because the type of passageways that are shown through the insert bodies 42 and 46 are of the type shown in the embodiment of FIGS. 7-9, the insert bodies 42 and 46 must be constructed of a malleable or compliant material. One or two heads can also be provided on the insert bodies of alternative embodiments of the invention.

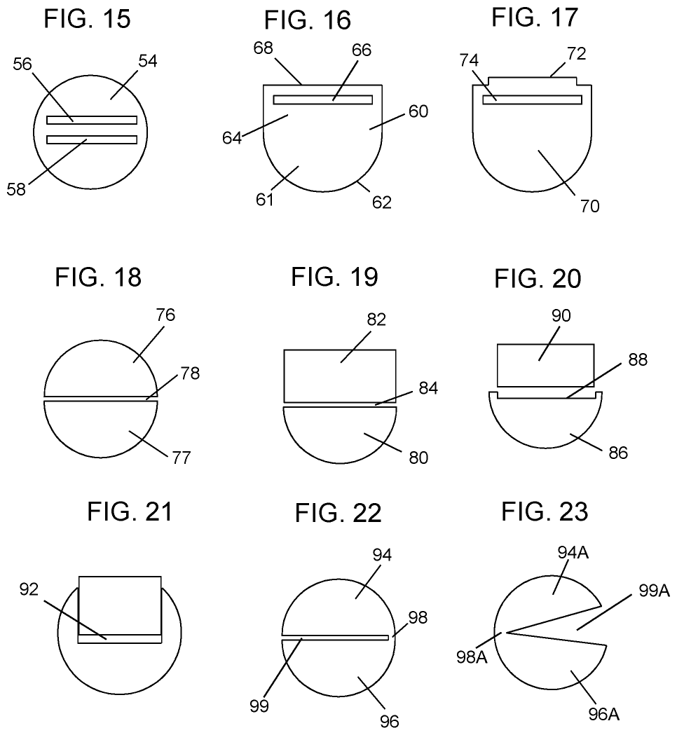

FIGS. 15 through 23 show cross sectional end views of examples of alternative embodiments of the invention. All of them have semi-cylindrical bottom surfaces which have a standard dimensions for seating conformingly in the socket of a tulip. Those with a circular periphery can be clamped in tulips with either a saddle or slot type socket or in tulips with a lateral, circular hole socket. As the following alternative embodiments of the invention are described, it will become apparent that there are many alternative structures with diverse configurations that can embody the invention. Most embodiments of the invention can be provided with one or two heads of the type described above. Insert bodies can be constructed with various cross sectional configurations. They can have one or more passageways. The passageways can be a hole as previously described. Passageways can also be a channel or other space between split, multiple components of an insert body that has matingly engageable components that fit together with their surfaces coinciding when placed against each other so they can be clamped together with a tether between them to grip the tether. Some are constructed of a malleable deformable material and some can be rigid.

FIG. 15 illustrates an insert body 54 which has an exterior configuration the same as the insert body 32 of FIGS. 7-9. The insert body 54 is formed of a malleable material and has two passageways 56 and 58. The second passageway 58 is formed through the insert body 54 transversely of the clamping screw's direction of travel when it is rotated, just like the orientation of the first passageway 56. The reason for providing a second passageway 58 is so that the surgeon can insert an end of the tether through one passageway in one direction and then insert the same end through the second passageway in the reverse direction. As a result the tether can be more strongly secured to the insert body 54 when the insert body 54 is clamped in the tulip socket and deformed against the tether.

FIG. 16 illustrates an insert body 60 having a lower portion 61 that is a semi-cylinder with a semi-cylindrical bottom surface 62 and an upper portion 64 that is a rectangular prism. The two portions are blended together to form the one piece insert body 60 with a lateral passageway 66. The insert 60 is only suitable for a saddle or slot type of tulip but its upper surface 68 can be engaged by the clamping screw of a tulip. It must be formed by a sufficiently malleable material.

FIG. 17 shows an insert body 70 that is like the insert body 60 of FIG. 16 but additionally has a projection, preferably in the form of a boss 72. The boss 72 is spaced above but in alignment or registration with the passageway 74 and is smaller than the entire width of the insert body 70. The boss 72 is an example of a projection that serves to concentrate the compressive force of a clamping screw that is located above the passageway 74 in order to concentrate the deformation of the insert body at the passageway 74.

FIGS. 18-21 show examples of insert bodies that comprise at least two matingly engageable components with a passageway that is a space between at least two of those components. At least two of the matingly engageable components are clampable together at mating, interfacing surfaces by a tulip's clamping screw. Clamping the components together forces their mating surfaces against opposite sides of a tether that is positioned between two of the components and thereby causes the interfacing surfaces to securely grip the tether within the insert body.

The insert body of FIG. 18 comprises two solid half cylinders 76 and 77 that have a passageway 78.

The insert body of FIG. 19 comprises a lower part that is a solid half cylinder 80 and an upper part 82 having the solid geometrical shape of a rectangular prism 82. Its passageway 84 is the tether-receiving gap between them.

FIG. 20 shows an insert body that is similar to the insert body of FIG. 19 but its lower semi-cylindrical component 86 additionally has a channel as its passageway 88. The upper component of its insert body is also a rectangular prism 90.

FIG. 21 shows an insert body that is like the insert body of FIG. 20 except that its lower component has a deeper channel for its passageway 92.

FIGS. 22 and 23 show examples of insert bodies that are each a unitary body having at least two lobes 94 and 96 and 94A and 96A respectively. The two lobes are joined together at respective hinge portions 98 and 98A. They each have a space between the lobes that forms respective passageways 99 and 99A. The space between the lobes is bordered by matingly engageable lobe surfaces between the lobes that are configured to be pressed together in mating engagement by rotation of a clamping screw into gripping contact against the tether.

The preferred materials for constructing the insert body of an embodiment of the invention are the materials known in the prior art for construction of surgical implant devices. These are most commonly a titanium alloy where malleability is not needed or commercially pure titanium where malleability is needed. Alternatively, for some applications the insert body can be constructed of a polymer that is approved for surgical implantation such as polyether ether ketone (PEEK).

### REFERENCE NUMBER LISTING

10 tulip
12 threaded shank of pedicle screw
14 socket of tulip
16 end openings of tulip
18 clamping screw
20 support rod
22 rod-to-rod connector
24 tulips of rod-to-rod connector
26 socket of rod-to-rod connector
27 rod in rod-to-rod connector 31 (FIG. 12)
28 end openings of rod-to-rod connector (FIG. 5)
29 tulips (FIGS. 11 & 12)
30 clamping screws of rod-to-rod connector (FIG. 6)
31 rod-to-rod connector (FIGS. 11 & 12)
32 insert body (FIGS. 7-12)
33 socket (FIGS. 11 & 12)
34 passageway (FIGS. 7-12)
35 bottom surface of insert body (FIGS. 10-12)
36 end openings of tulip 29 (FIGS. 11 & 12)
38 clamping screw (FIGS. 11 & 12)
40 tether (FIG. 10)
42 insert body (FIG. 13)
44 head of insert body (FIG. 13)
46 insert body (FIG. 13)
48-50 heads of insert body (FIG. 14)
52 passageway (FIG. 14)
54 insert body
56 first passageway
58 second passageway
60 insert body
62 semi-cylindrical bottom surface
64 upper portion 64
66 lateral passageway 66
68 upper surface 68
70 insert body
72 boss 72
74 passageway
76, 77 two solid half cylinders
78 passageway
80 solid half cylinder 80
82 upper part having a solid geometrical shape of a rectangular prism
84 passageway
86 lower semi-cylindrical component
88 passageway
90 rectangular prism
92 passageway
94,96 two lobes (FIG. 22)
94A,96A two lobes (FIG. 23)
98,98A hinge portions (FIGS. 22 & 23)
99,99A passageways (FIGS. 22 & 23)

This detailed description in connection with the drawings is intended principally as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention and that various modifications may be adopted without departing from the invention or scope of the following claims.

1. (canceled)

2. (canceled)

3. (canceled)

4. (canceled)

5. (canceled)

6. A spinal fixation construct according to claim 16 wherein the insert body comprises two solid half cylinders.

7. A spinal fixation construct according to claim 16 wherein the insert body comprises a solid half cylinder and a solid rectangular prism.

8. (canceled)

9. A spinal fixation construct according to claim 17 wherein the passageway is a hole through the insert body transversely of the clamping screw's direction of travel when rotated.

10. A spinal fixation construct according to claim 9 wherein a second hole is formed through the insert body transversely of the clamping screw's direction of travel when rotated.

11. A spinal fixation construct according to claim 17 wherein the insert body comprises a unitary body having at least two lobes that are joined together and have a space between the lobes forming the passageway, the space being between matingly engageable lobe surfaces that are configured to be pressed together by rotation of the clamping screw into gripping contact against the artificial ligament tether.

12. A spinal fixation construct comprising:
(a) a rod-to-rod connector comprising at least two tulips rigidly connected together as a unitary body, each tulip including a socket having opposite end openings and configured in size and shape to receive a spinal longitudinal support rod, each tulip further including a clamping screw threadedly engaged to the tulip and configured to clamp a support rod in its socket;
(b) a spinal longitudinal support rod clamped in the socket of a first tulip of the tulips;
(c) an artificial ligament tether; and
(d) an insert far convertibly anchoring the artificial ligament tether in a second tulip of said tulips, the insert comprising an insert body having an exterior configured in size and shape to conformingly seat against a surface of the socket of the second tulip, the insert body having a passageway extending entirely through the body in a direction to extend between the end openings of the second tulip and configured to receive the artificial ligament tether, wherein rotation of the clamping screw of the second tulip translates the clamping screw against the body and clamps the artificial ligament tether, which is inserted through the passageway, to the body and clamps the body to the second tulip.

13. A spinal fixation construct according to claim 12 wherein the second tulip and the insert body have mating, cylindrically contoured surfaces that are clampable in the socket of the second tulip against each other by the clamping screw of the second tulip with the cylindrically contoured surface of the insert body extending between the end openings of the second tulip.

14. A spinal fixation construct according to claim 13 wherein the insert body has a head at an end that is enlarged beyond the size of the opposite end openings of the second tulip for preventing the insert body from sliding out of the second tulip's socket.

15. A spinal fixation construct according to claim 14 wherein the insert body has a first head at a first end and a second head at an opposite second end, both heads being enlarged beyond the opposite end openings of the second tulip, the heads being spaced apart by a distance at least as far as the distance between the end openings of the second tulip for preventing the insert body from sliding out of the second tulip's socket.

16. A spinal fixation construct according to claim 13 wherein the insert body comprises at least two matingly engageable components and wherein the passageway is a space between at least two components, at least two of the matingly engageable components being clampable together by the second tulip's clamping screw against an artificial ligament tether positioned between two of the components to grip the artificial ligament tether.

17. A spinal fixation construct according to claim 13 wherein the insert body is constructed of a malleable material that is deformable by rotation of the second tulip's clamping screw into gripping contact against an artificial ligament tether.

18. A method for surgically preparing a spinal fixation construct, the construct including a tulip with a socket having opposite end openings and configured in size and shape to receive in the socket a spinal longitudinal support rod, the method including:
(a) installing and attaching a manually removable tether anchor in the tulip socket of the construct;
(b) fixing a tether to the tether anchor and to a vertebra;
(c) in a subsequent surgery, detaching and removing the tether and the tether anchor from the tulip; and
(d) installing a longitudinal support rod in the socket of the tulip.

19. A method according to claim 18 wherein the tulip has a clamping screw threadedly engaged to the tulip and configured to clamp the support rod in the socket the method further including:
installing and attaching the manually removable tether anchor in the tulip socket by rotating the clamping screw to clamp the tether anchor in the tulip socket.
